(19) United States
(12) Patent Application Publication
(10) Pub. No.: US 2006/0135960 A1
(43) Pub. Date: Jun. 22, 2006

(54) Bone screw comprising a tangential cutting edge

(76) Inventors: Chad Munro, Mabou (CA); Peter Senn, Waldenburg (CH)

Correspondence Address: JONES DAY, 222 EAST 41ST STREET, NEW YORK, NY, 10017-6702, US

(21) Appl. No.: 10/518,866

(22) PCT Filed: Jun. 21, 2002
(86) PCT No.: PCT/CH02/00337

§ 371 Date: Jan. 30, 2006

(51) Int. Cl. A61F 2/36

(52) U.S. Cl. 606/104

(57) ABSTRACT

The invention relates to a bone screw having a longitudinal axis and a threaded shaft which has an external thread of an external diameter D_{A}, a front thread end and a thread profile. The thread profile has a front thread flank that is oriented towards the front thread end. The front thread flank comprises a flank section that has a 90° offset in relation to the front thread flank, thereby forming a tangential cutting edge on the front thread end.

### TECHNICAL FIELD

The invention relates generally to a bone screw and a device comprising a bone screw and a tubular bone blade.

### BACKGROUND OF THE INVENTION

Bone screws used in osteosynthesis are frequently provided with self-cutting threads, so that, during the implantation of the bone screw, the thread does not have to be cut in the bone by the surgeon in a separate step after the drilling. In the case of known self-cutting bone screws, this self-cutting property may be achieved by grooves, which are disposed at the front end of the bone screw parallel to the longitudinal axis of the bone screw. Other bone screws are provided with self-forming threads. In the case of these self-forming external threads, the external diameter of the thread decreases towards the front end of the bone screw. In the case of self-cutting as well as in the case of self-forming external threads, the load-carrying capacity of the thread decreases towards the front end of the bone screw.

A bone screw with a self-forming thread is also discussed in the U.S. Pat. No. 5,061,135 to Pritchard. The Pritchard bone screw has an external thread, the front threads of which at the tip of the screw have a decreasing profile height towards the tip of the screw. A disadvantage of this bone screw is that at the threads furthest to the front, which have a lower profile height, the bone screw is held less firmly, particularly in osteoporotic bends.

### SUMMARY OF THE INVENTION

The present invention is to provide a remedy for the above-discussed disadvantage. An object of the present invention is to create a to create a bone screw, the front end of the thread of which has a tangential cutting edge, so that the external thread ensures a maximum hold up to the thread furthest to the front. This constant load-carrying capacity of the thread up to the front end of the bone screw is advantageous especially if the bone screw is used in the proximal femur for fixing the head of the hip joint.

The present invention accomplishes the objective set out above with a bone screw having a longitudinal axis comprising a threaded shaft. The threaded shaft comprises an external thread with an external diameter D_{A}, a front threaded end and a thread profile, wherein the thread profile has a front threaded flank which is directed towards the front threaded end, and a rear threaded flank. The bone screw also has a front end, and a rear end, the rear end able to accommodate a tool. The external thread at the front end of the thread comprises a flank piece, which is angled with respect to threaded flanks, so that a tangential cutting edge is formed thereby at the front end of the thread.

An advantage achieved by the present invention is that, due to the inventive bone screw, the external thread is provided only on the first thread with a flank piece, which is angled with respect to the front threaded flank and that therefore the thread profile is reduced in width only there. Due to this configuration of the front end of the thread, a better retention can be achieved especially in the spongiosa of osteoporotic bends. This is of great importance especially for bone screws, which are to be used at osteoporotic long bones, such as an osteoporotic, proximal femur or an osteoporotic proximal humerus.

Due to the configuration of the angle of the flank piece at the front thread, the point angle of the tangential cutting edge can be selected relative to the longitudinal axis and to the flank angle of the threaded profile respectively.

In a preferred embodiment of the inventive bone screw, the angle α between the flank piece and the longitudinal axis of the bone screw is between 40° and 110° and typically is 80°.

In a different embodiment of the inventive bone screw, the threaded shaft has a constant profile height, as a result of which the hold of the bone screw in the bone can be improved even further.

The inventive bone screw may also have a multiple external thread, preferably a double external thread.

In a further embodiment of the inventive bone screw, the external thread has a thread pitch x of between 1 mm and 7 mm and preferably of between 1.5 mm and 4.0 mm.

In yet another embodiment of the inventive bone screw with an external thread with n threads, the thread pitch is X=nx.

In a different embodiment of the inventive bone screw, the external diameter D_{A} of the external thread is between 7 and 14 mm and preferably between 10 mm and 14 mm. Typically, the external diameter D_{A}=12 mm, so that, by this configuration of the external thread, it can be achieved that the bone screw can be anchored especially in the spongiosa of a bone and is not suitable as a corticalis screw.

In a further embodiment of the inventive bone screw, the height of the profile H is between 0.5 mm and 5.0 mm and preferably between 2.5 mm and 4.5 mm.

In a different embodiment yet of the inventive bone screw, the threaded profile is provided with a flank angle β of between 5° and 160° and preferably of between 60° and 90°. This flank angle β can also be variable in a cross-sectional area of the bone screw, parallel to the longitudinal axis, when viewed over the height of the profile.

In one embodiment of the inventive device, the latter comprises a bone screw of one of the embodiments listed above and a tubular bone blade with a central borehole, which is coaxial with the longitudinal axis of the bone screw. The front end of the bone screw protrudes coaxially beyond the bone blade, so that, by rotating the bone screw about its longitudinal axis, the bone blade is pulled into a bone. Moreover, the bone screw, which can be rotated about its longitudinal axis, is mounted in the central borehole of the bone blade and secured axially at least against being pulled at of the bone blade in the direction of the front end. The advantage of this device lies therein that the bone blade does not have to be knocked into the bone and, instead, can be pulled into it by means of the bone screw.

Other objectives and advantages, in addition to those discussed above, will become apparent to those skilled in the art during the course of the description of the embodiments of the invention which follows. In the description, reference is made to accompanying drawings, which form a part thereof, and which illustrate examples of the invention. Such examples, however, are not exhaustive of the various embodiments of the invention, and therefore, reference is made to the claims that follow the description for determining the scope of the invention.

### BRIEF DESCRIPTION OF THE DRAWINGS

FIG. 1 shows a side view of the preferred embodiment of the inventive bone screw;

FIG. 2 shows a front view of the preferred embodiment of the inventive device;

FIG. 3 shows a side view of a different embodiment of the inventive bone screw;

FIG. 4 shows a front view of the embodiment of the inventive device, shown in FIG. 3;

FIG. 5 shows a side view of a further embodiment of the inventive bone screw;

FIG. 6 shows a view of an embodiment of the inventive bone screw during the fixation of fracture of the neck of the proximal femur;

FIG. 7 shows a longitudinal section through a further embodiment of the inventive bone screw; and

FIG. 8 shows a perspective view of an embodiment of the inventive device with a bone screw.

### DESCRIPTION OF THE PREFERRED EMBODIMENTS

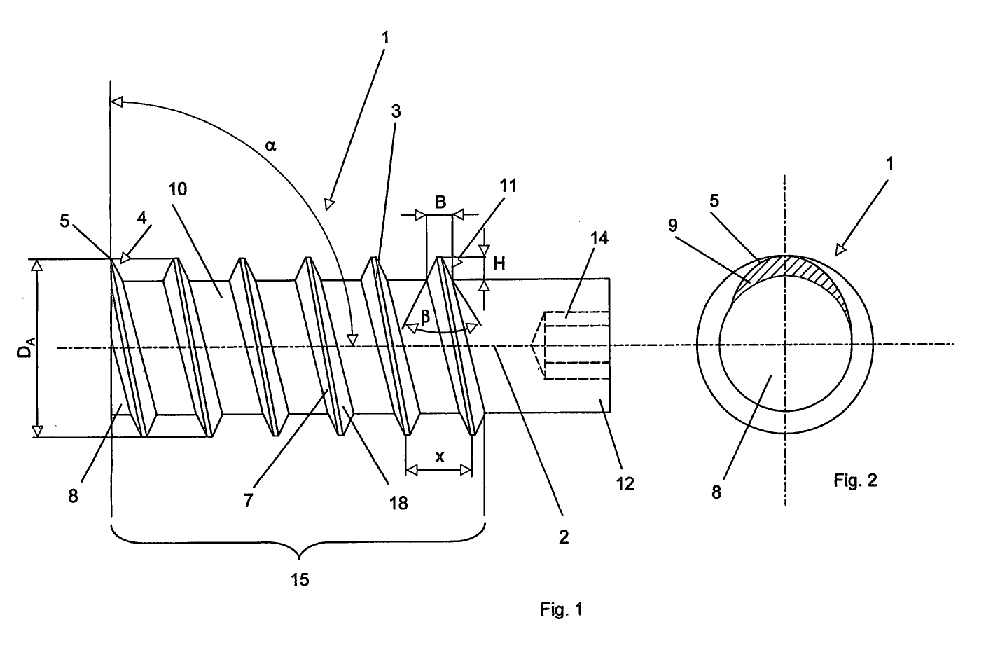

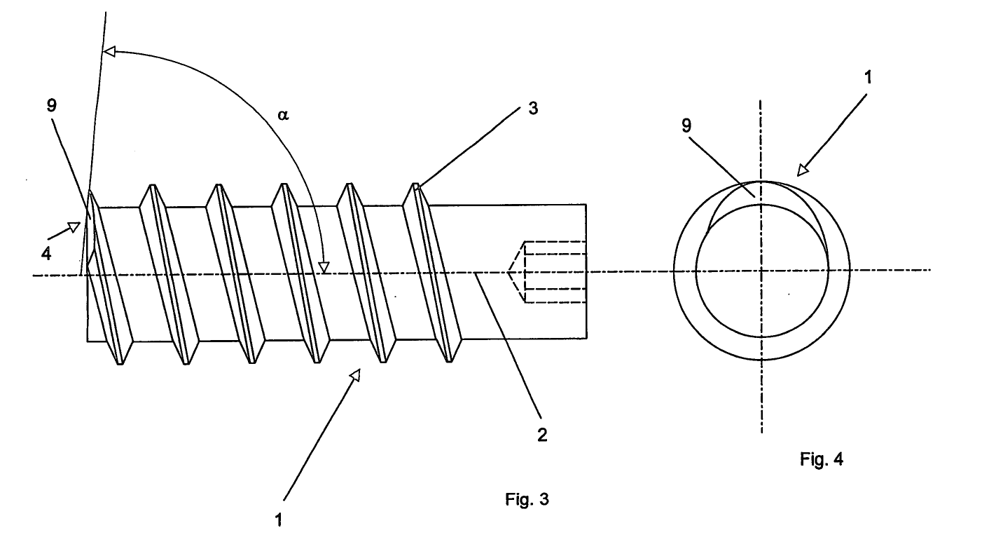

The bone screw 1 is shown in FIGS. 1 and 2 includes a front (distal) end 8, a rear (proximal) end 12, and threaded shaft 15 with an external thread 3 with the thread pitch x. At the rear end 12 of the bone screw 1, means 14 for accommodating a screwdriver, which are shown here, by way of example, as a hexagon socket, are mounted coaxially with the longitudinal axis 2 of the bone screw 1. At the front end 8 of the bone screw 1, the latter is constructed flat and orthogonal to the longitudinal axis 2. In the embodiment shown here of the bone screw 1, the front threaded end 4 of the external thread 3 coincides with the front end 8 of the bone screw 1. Furthermore, the external thread 3 at the front end 4 includes a flank piece 9 (FIG. 2), which is angled with respect to the front threaded flank 7, which is directed towards the front end 8 of the bone screw 1, and with respect to the rear threaded flank 18. By these means, a tangential cutting edge 5 is formed on the first thread at the front end 4 of the threaded section. Due to the configuration of the angle of the flank piece at the front thread, the point angle of the tangential cutting edge can be selected relative to the longitudinal axis and to the flank angle of the threaded profile respectively. Together with the longitudinal axis 2, the flank piece 9 encloses an angle α, which is 90° here. The thread profile 11 has a profile height H, a flank angle β and a width B at the thread core 10.

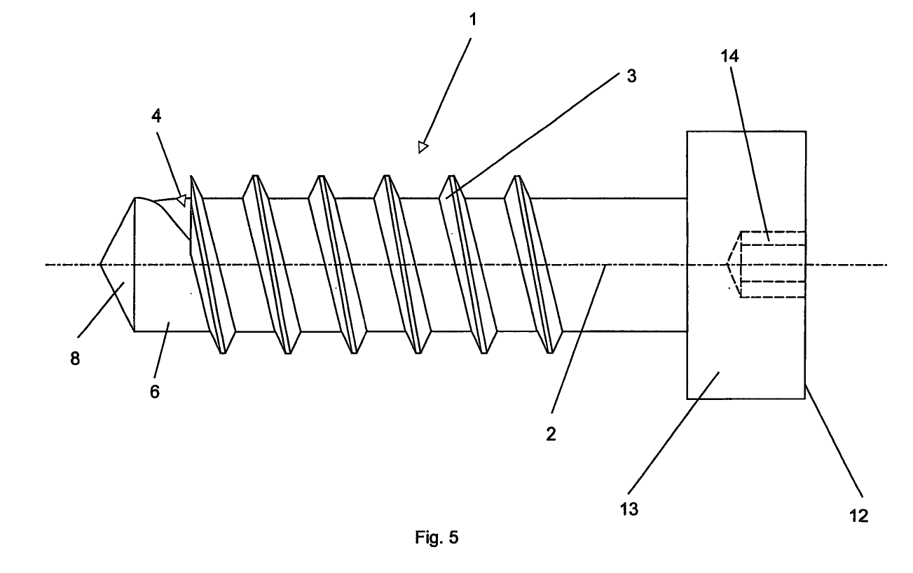

The embodiment of the bone screw 1, shown in FIGS. 3 and 4, differs from the embodiment, shown in FIGS. 1 and 2 only in that the flank piece 9, disposed at the front end 4 of the threaded section at the external thread 3, encloses an angle α of 85° with the longitudinal axis 2.

In another embodiment of the bone screw, the angle α between the flank piece and the longitudinal axis of the bone screw is between about 40° and about 110° and typically is about 80°. The angle α may also be between about 85° and about 95°, and more preferably between about 88° and about 91°

FIG. 5 depicts another embodiment of the bone screw 1. The bone screw 1 comprises a drill point 6 at its front end 8 and a screw head 13 at its rear end 12. The drill point 6 may be constructed continuously up to a front end 4 of the external thread 3. At the screw head 13, parallel to the longitudinal axis 2, means 14 are provided for accommodating a screwdriver, which may be constructed here, by way of example, as a hexagon socket.

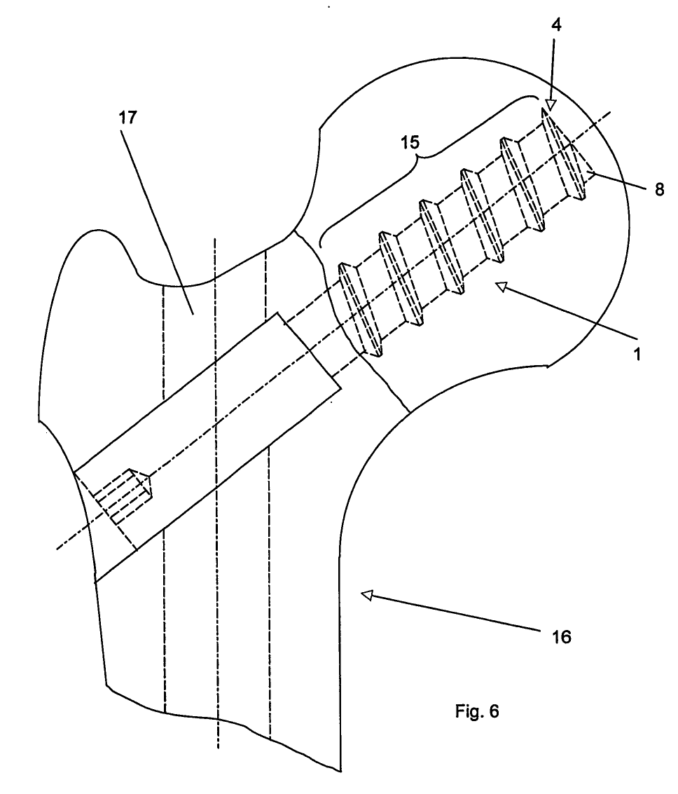

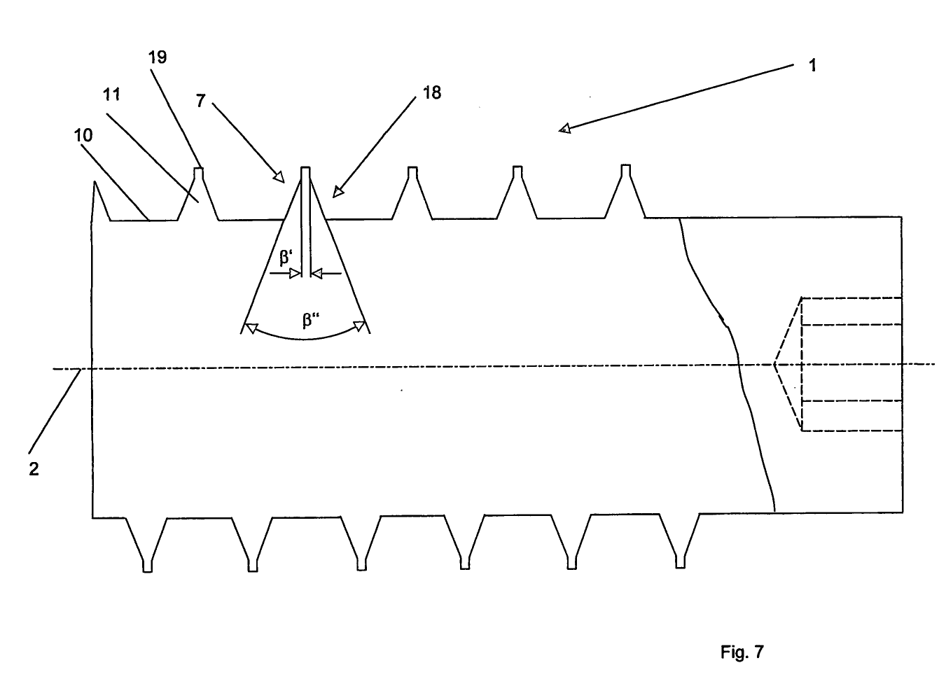

FIG. 6 depicts an embodiment of the bone screw 1 together with a medullary pin 17 as fixation screw in the event of a bone fracture at the proximal femur 16. Because the threaded profile 11 (FIG. 1) of all threads over the whole length of the threaded shaft 15 up to the front end 4 of the threaded section has constant dimensions, the bone screw 1 can be inserted with a short threaded shaft 15. Because the threaded profile 11 (FIG. 1) is not reduced in size at the front end 8 of the bone screw 1, the bone screw 1 may be held better in the vicinity of the front end 8 of the bone screw 1.

FIG. 7 shows a longitudinal section parallel to the longitudinal axis 2 of the bone screw 1. The embodiment of the bone screw 1, shown here, differs from the previous embodiment shown only in that the front and rear threaded flanks 7, 18, viewed in a cross section parallel to the longitudinal axis 2 of the screw 1 have a constant flank angle β. The threaded profile 11 comprises threaded flanks 7; 18, which have an angle β′=0° at the thread point 19 and an angle β″=80° at the thread core 10.

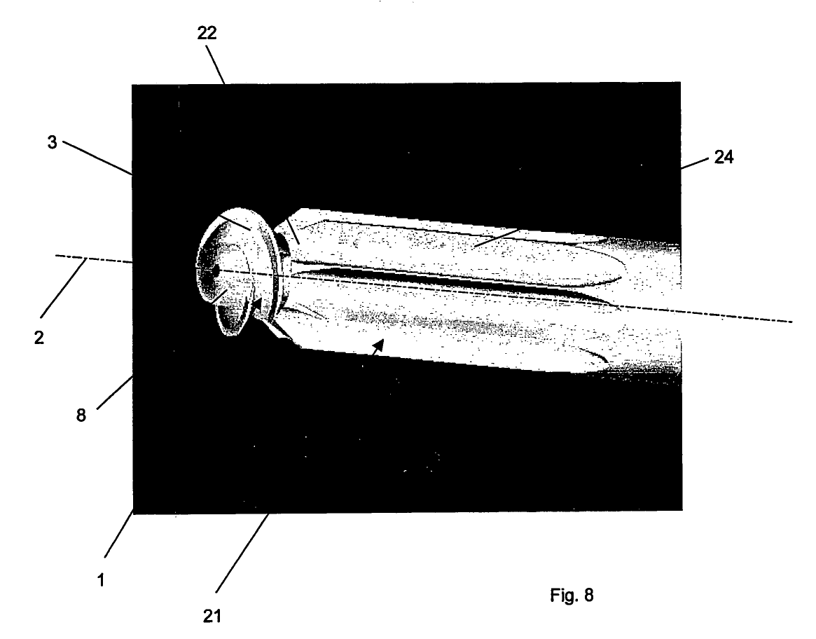

FIG. 8 shows an embodiment which comprises a bone screw 1 of one of the embodiments discussed above and a tubular bone blade 21 with a central borehole, coaxial with the longitudinal axis 2 of the bone screw 1. The bone screw 1 protrudes with its front end 8 and its external thread 8 coaxially beyond the front end 22 of the bone blade 21, so that, by rotating the bone screw 1 about its longitudinal axis 2, the bone blade 21 is pulled into a bone. The bone screw 1 may be rotated about its longitudinal axis 2, is mounted in the central borehole of the bone blade 21 and secured axially at least against being pulled out of the bone blade 21 in the direction of the front end 22. Furthermore, the bone screw 1 is constructed at its rear end 12 (FIG. 1) with means 14 for accommodating a screwdriver.

In another embodiment of the bone screw 1, the threaded shaft 15 has a constant profile height, as a result of which the hold of the bone screw 1 in the bone can be improved even further.

The bone screw 1 may also have a multiple external thread, preferably a double external thread.

In a further embodiment of the bone screw 1, the external thread has a thread pitch x of between about 1 mm and about 7 mm and preferably of between about 1.5 mm and about 4.0 mm.

In yet another embodiment of the bone screw 1 with an external thread 3 with n threads, the thread pitch is X=nx.

In a different embodiment of the bone screw 1, the external diameter D_{A} of the external thread 3 is between about 7 and about 14 mm and preferably between about 10 mm and about 14 mm. Typically, the external diameter D_{A} equals about 12 mm, so that, by this configuration of the external thread 3, the bone screw 1 may be anchored especially in the spongiosa of a bone and is not suitable as a corticalis screw.

In a further embodiment of the bone screw 1, the height of the profile H is between about 0.5 mm and about 5.0 mm and preferably between about 2.5 mm and about 4.5 mm.

In a different embodiment of the bone screw 1, the threaded profile is provided with a flank angle β of between about 5° and about 160° and preferably of between about 60° and about 90°. This flank angle β can also be variable in a cross-sectional area of the bone screw 1, parallel to the longitudinal axis 2, when viewed over the height of the profile.

1. A bone screw having a longitudinal axis comprising:
a threaded shaft, which comprises an external thread with an external diameter D_{A}, a front threaded end and a thread profile, wherein the thread profile as a front threaded flank which is directed towards the front threaded end, and a rear threaded flank;
a front end; and
a rear end, the rear end able to accommodate a tool,
wherein the external thread at the front end of the thread comprises a flank piece, which is angled with respect to threaded flanks, so that a tangential cutting edge is formed thereby at the front end of the thread.

2. A bone screw according to claim 1, wherein flank piece encloses an angle α of between about 40° and about 110° with the longitudinal axis.

3. A bone screw according to claim 2, wherein the angle α is between about 85° and about 95°, and more preferably between about 88° and about 91°.

4. A bone screw according to claim 1, wherein the threaded profile has a constant profile height H.

5. A bone screw according to claim 1, wherein the external thread is configured as a multiple thread, preferably as a double thread.

6. A bone screw according to claim 1, wherein the external thread has a thread pitch x of between about 1 mm and about 7 mm, and preferably of between about 1.5 mm and about 4.0 mm.

7. A bone screw according to claim 6, wherein the external thread has n threads and a thread pitch X=nx.

8. A bone screw according to claim 1, wherein the external diameter D_{A} of the external thread is between about 7 and about 14 mm and preferably between about 10 mm and about 14 mm.

9. A bone screw according to claim 1, wherein the height H of the thread profile is between about 0.5 mm and about 5.0 mm, and more preferably between about 2.5 mm and about 4.5 mm.

10. A bone screw according to claim 1, wherein the thread profile has a flank angle β between about 5° and about 160°, and more preferably between about 60° and about 90°.

11. A bone screw according to claim 1, wherein the thread profile has a variable flank angle β in a cross-sectional area parallel to the longitudinal axis of the bone screw.

12. An assembly comprising:
a bone screw having a front end and a rear end forming a longitudinal axis, wherein the bone screw further includes a threaded shaft, which comprises an external thread with an external diameter D_{A}, a front threaded end and a thread profile, wherein the thread profile has a front threaded flank which is directed towards the front threaded end, and a rear threaded flank;
a tubular bone blade with a central borehole which is coaxial with the longitudinal axis of the bone screw,
wherein the front end of the bone screw protrudes coaxially beyond the tubular bone blade and the bone screw being rotatable about the longitudinal axis in the central borehole, and
wherein the external thread of the bone screw at the front end of the thread comprises a flank piece, which is angled with respect to threaded flanks, so that a tangential cutting edge is formed thereby at the front end of the thread.
